(19) United States
(12) Patent Application Publication
(10) Pub. No.: US 2006/0079513 A1
(43) Pub. Date: Apr. 13, 2006

(54) Methods and compositions including methscopolamine nitrate

(76) Inventor: David M. Preston, Chapel Hill, NC (US)

Correspondence Address: HUTCHISON & MASON PLLC, PO BOX 31686, RALEIGH, NC, 27612, US

(21) Appl. No.: 10/964,252

(22) Filed: Oct. 13, 2004

(51) Int. Cl. A61K 31/5513; A61K 31/46; A61K 31/44

(52) U.S. Cl. 514/221; 514/357; 514/304

(57) ABSTRACT

Therapeutic pharmaceutical compositions are provided that include an anticholinergic agent and a sedative agent. Particularly preferred anticholinergic agents include anticholinergic agents which do not substantially cross the blood-brain barrier. Methscopolamine nitrate is the preferred anticholinergic agent. The sedative agent may be a benzodiazepine or a barbiturate. Particularly, the sedative agent may be chlordiazepoxide hydrochloride, diazepam, or phenobarbital. Various methods using the compositions to alleviate gastrointestinal disorders or symptoms thereof are also provided.

### FIELD

The present invention is directed to therapeutic agents for the treatment of gastrointestinal disorders and methods for administering those agents. The invention generally relates to therapeutic pharmaceutical compositions including an anticholinergic agent and a sedative agent. The therapeutic pharmaceutical compositions may be used in methods for treating gastrointestinal tract disorders or conditions or symptoms of such disorders or conditions.

### BACKGROUND

Anticholinergic agents are typically antagonistic to the action of parasympathetic or other cholinergic nerve fibers. Generally, anticholinergic agents block or inhibit the effects of acetylcholine which is produced by the body and is responsible for certain nervous system activities. Various anticholinergic compounds are known which have a variety of effects on the human body depending on the particular structure of the compound. Anticholinergic agents derived from the belladonna alkaloids may produce a number of effects in the body, including relief from spasms of the gastrointestinal tract, the bladder and the biliary tract. Belladonna alkaloid anticholinergic compounds include the tertiary amines atropine, hyoscyamine and scopolamine, which are believed to cross the blood brain barrier and exert an effect on the central nervous system. The effects on the central nervous system may be a negative consequence of the use of these anticholinergic compounds, causing a variety of unwanted side effects.

Quaternary ammonium anticholinergic agents have been derived from the belladonna alkaloids by such modifications as, for example, by the addition of a second methyl group to the nitrogen. Such quaternary ammonium anticholinergic agents are believed to not cross the blood brain barrier, and, thus, do not exert the same effect on the central nervous system as the belladonna alkaloids from which these compounds may be derived.

Clidinium bromide is a quaternary ammonium anticholinergic agent. It has been used in Librax™, which contains clidinium bromide and chlordiazepoxide hydrochloride, a benzodiazepine. Librax™ has been prescribed for the treatment of a variety of gastrointestinal disorders such as peptic ulcer, irritable bowel syndrome and acute enterocolitis. The Food and Drug Administration, FDA, however, has questioned the effectiveness of this compound. In addition, while clidinium bromide may not have the effects of the belladonna alkaloids on the central nervous system, it is postulated that clidinium bromide has the potential to cause liver toxicity in some patients.

In view of the foregoing, therapeutic pharmaceutical compositions useful for the treatment of gastrointestinal tract disorders or symptoms thereof and methods for administration of the therapeutic pharmaceutical compositions are still needed. Thus, there is still a need in the art for a formulation of anticholinergic agents which is substantially free of the disadvantages, defects and limitations of the formulations disclosed in the art.

### SUMMARY

In accordance with the foregoing, there are provided by the embodiments of the present invention therapeutic pharmaceutical preparations consisting essentially of a therapeutically effective amount of an anticholinergic agent and a sedative agent whereby the combination of anticholinergic agent and sedative agent alleviates one or more gastrointestinal disorders or one or more symptom thereof.

In one embodiment, a therapeutic pharmaceutical composition is provided consisting essentially of a therapeutically effective amount of a blended mixture of an anticholinergic agent comprising methscopolamine nitrate and a sedative agent comprising chlordiazepoxide hydrochloride. In a preferred embodiment, the ratio of the methscopolamine nitrate to the chlordiazepoxide hydrochloride is about 0.5:1 to about 1:1. In another preferred embodiment, the methscopolamine nitrate is present in an amount of about 2.0 to about 5.0 mg per dose (about 8.0 mg to about 20.0 mg per day) and the chlordiazepoxide hydrochloride is present in an amount of about 5.0 mg per dose (about 20.0 mg per day). The therapeutic pharmaceutical composition preferably is administered orally in an immediate release form given four times a day or is in a sustained release preparation given less than four times a day, and is available in powder form for delivery to a patient in need of the therapeutic pharmaceutical composition.

In another embodiment, a therapeutic pharmaceutical composition is provided consisting essentially of a therapeutically effective amount of a blended mixture of an anticholinergic agent comprising methscopolamine nitrate and a sedative agent comprising diazepam. In a preferred embodiment, the methscopolamine nitrate is present in an amount of about 2.0 to about 5.0 mg per dose (about 8.0 mg to about 20.0 mg per day) and the diazepam is present in an amount of about 2.0 to about 5.0 mg per dose (about 8.0 mg to about 20.0 mg per day). The therapeutic pharmaceutical composition preferably is administered orally in an immediate release form given four times a day or is in a sustained release preparation given less than four times a day, and is available in powder form for delivery to a patient in need of the therapeutic pharmaceutical composition.

In a further embodiment, a therapeutic pharmaceutical composition is provided consisting essentially of a therapeutically effective amount of a blended mixture of an anticholinergic agent comprising methscopolamine nitrate and a sedative agent comprising phenobarbital. In a preferred embodiment, the methscopolamine nitrate is present in an amount of about 2.0 to about 5.0 mg per dose (about 8.0 mg to about 20.0 mg per day) and the phenobarbital is present in an amount of about 16 to about 32 mg per dose (about 64 mg to about 128 mg per day). The therapeutic pharmaceutical composition preferably is administered orally in an immediate release form given four times a day or is in a sustained release preparation given less than four times a day, and is available in powder form for delivery to a patient in need of the therapeutic pharmaceutical composition.

In yet another embodiment, a method of alleviating at least one gastrointestinal disorder or at least one symptom of a gastrointestinal disorder in a human patient is provided, the method comprising administering to the patient a therapeutic pharmaceutical composition consisting essentially of a therapeutically effective amount of a blended mixture of methscopolamine nitrate and chlordiazepoxide hydrochloride. In a preferred embodiment, the ratio of the methscopolamine nitrate to the chlordiazepoxide is about 0.5:1 to about 1:1. In another preferred embodiment, the methscopolamine nitrate is present in an amount of about 2.0 to about 5.0 mg per dose (about 8.0 mg to about 20.0 mg per day) and the chlordiazepoxide hydrochloride is present in an amount of about 5.0 mg per dose (about 20 mg per day). The therapeutic pharmaceutical composition preferably is administered orally in an immediate release form given four times a day or is in a sustained release preparation given less than four times a day, and is available in powder form for delivery to a patient in need of the therapeutic pharmaceutical composition.

In yet a further embodiment, a method of alleviating at least one gastrointestinal disorder or at least one symptom of a gastrointestinal disorder in a human patient is provided, the method comprising administering to the patient a therapeutic pharmaceutical composition consisting essentially of a therapeutically effective amount of a blended mixture of methscopolamine nitrate and diazepam. In a preferred embodiment, the methscopolamine nitrate is present in an amount of about 2.0 to about 5.0 mg per dose (about 8.0 mg to about 20.0 mg per day) and the diazepam is present in an amount of about 2.0 to about 5.0 mg per dose (about 8.0 mg to about 20.0 mg per day). The therapeutic pharmaceutical composition preferably is administered orally in an immediate release form given four times a day or is in a sustained release preparation given less than four times a day, and is available in powder form for delivery to a patient in need of the therapeutic pharmaceutical composition.

In a further embodiment, a method of alleviating at least one gastrointestinal disorder or at least one symptom of a gastrointestinal disorder in a human patient is provided, the method comprising administering to the patient a therapeutic pharmaceutical composition consisting essentially of a therapeutically effective amount of a blended mixture of methscopolamine nitrate and phenobarbital. In a preferred embodiment, the methscopolamine nitrate is present in an amount of about 2.0 to about 5.0 mg per dose (about 8.0 mg to about 20.0 mg per day) and the phenobarbital is present in an amount of about 16 to about 32 mg per dose (about 64 mg to about 128 mg per day). The therapeutic pharmaceutical composition preferably is administered orally in an immediate release form given four times a day or is in a sustained release preparation given less than four times a day, and is available in powder form for delivery to a patient in need of the therapeutic pharmaceutical composition.

### DETAILED DESCRIPTION

The present invention relates to methods and compositions for various uses, including the administration of anticholinergic agents with one or more sedative agents for the treatment of disorders or conditions or symptoms thereof relating to the gastrointestinal tract, including the stomach and/or intestines. It has been found that the deficiencies of the prior described pharmaceutical formulations of anticholinergic agents for the treatment of gastrointestinal disorders and/or symptoms thereof can be overcome by the use of methscopolamine nitrate as the anticholinergic agent with particular sedative agents in the pharmaceutical or therapeutic formulation. Methscopolamine nitrate may, in some embodiments, be used in the substantial absence of other active agents. Prior to describing this invention in further detail, however, the following terms will first be defined.

### DEFINITIONS

The phrase “alleviating a symptom of a gastrointestinal disorder” means reducing or eliminating the severity or the frequency of the symptom or both.

The phase “alleviating a gastrointestinal disorder” means reducing or eliminating one or more symptoms suffered by the patient due to one or more condition, illness, infection, or disease state involving the gastrointestinal tract, including, but not limited to the stomach and/or bowel. Exemplary gastrointestinal disorders include, but are not limited to, ulcer, bowel spasms, abdominal pain, bloating, cramps, inflammation of the stomach and/or intestines, irritable bowel syndrome, inflammatory bowel disease and the like.

A “disorder” includes any condition, illness, disease, infection or the like.

“Effective amount” or “therapeutically effective amount” means the amount needed for the desired therapeutic effect and includes any additional amount or overage of active ingredient deemed necessary in the formulation to provide the desired amount upon administration.

“Available for immediate delivery” means the therapeutic pharmaceutical composition is provided in a formulation allowing the blended mixture of anticholinergic agent and sedative agent to begin acting in a therapeutic manner substantially as soon as the agents become available in the body and/or bloodstream of the patient.

“Sustained release” means the therapeutic pharmaceutical composition is provided in a formulation such that the composition provides an initial therapeutic effect and also an ongoing or additional therapeutic release of therapeutic pharmaceutical composition or therapeutic effect over a desired period of time.

“Substantially no liver toxicity” means that a patient ingesting a therapeutic pharmaceutical composition consisting essentially of an anticholinergic agent and sedative agent according to embodiments disclosed herein does not experience a substantial increase in liver enzyme production associated with administration of the composition.

“Anticholinergic compounds” include compounds typically antagonistic to the action of parasympathetic or other cholinergic nerve fibers.

Methscopoloamine nitrate is an anticholinergic agent having the chemical name 3-oxa-9-azonlatricyclo [3.3.1.0] nonane, 7-(3-hydroxy-1-oxo-2-phenylpropoxy)-9,9-dimethyl-nitrate,[7(S)-1(1α,2β,4β,5α,7β)]. Methscopoloamine nitrate is a quaternary ammonium derivative of the anticholinergic scopolamine which possesses the peripheral actions of the belladonna alkaloids, but does not exhibit the central actions because of its lack of ability to cross the blood-brain barrier. Without being bound to any theory, methscopolamine nitrate, when administered according to the embodiments disclosed with a sedative agent, is believed to decrease parasympathetic tone in the gastrointestinal tract and slow gastrointestinal motility without substantial negative side effects, thus effectively providing the desired therapeutic effect in alleviating gastrointestinal disorders or symptoms thereof. Methscopolamine nitrate is also believed to substantially avoid the potential liver toxicity issues of other quaternary ammonium anticholinergic agents.

The amount of methscopolamine nitrate generally will be the equivalent of about 8 mg/day to about 20 mg/day. A typical dosage is about 2.0 mg to about 5.0 mg administered four times a day. The preferred dosage is about 2.5 mg administered orally four times a day.

The anticholinergic agent may be administered as the primary active agent or in the substantial absence of other active therapeutic agents, but preferably is administered in combination with a sedative agent as a blended mixture of anticholinergic agent and sedative agent. The sedative agent is an agent known to cause a sedating or tranquilizing effect on a mammal, i.e., having the capacity to depress the function of the central nervous system such that calming, relaxation or drowsiness is produced. Sedative agents useful in the present therapeutic pharmaceutical compositions may include benzodiazepines, or barbiturates, preferably, chlordiazepoxide hydrochloride, diazepam or phenobarbital.

The amount of chlordiazepoxide hydrochloride when used in the pharmaceutical composition typically will be about 5 mg administered four times a day. Thus, the typical dose will be about 20 mg a day. The amount of diazepam when used in the pharmaceutical composition typically will be about 2 to about 5 mg administered four times a day. Thus, the typical dosage of diazepam will be about 8 to about 20 mg a day. The amount of phenobarbital when used in the pharmaceutical composition typically will be about 16 to about 32 mg administered four times a day. Thus, the typical dosage of phenobarbital will be about 64 to about 128 mg a day. The dosages of the sedative agent may be adjusted according to the desired effect as is known in the art.

The therapeutic pharmaceutical compositions disclosed may include a number of other components for obtaining optimal delivery characteristics of the formulation depending on the desired method and form of administration of the therapeutic pharmaceutical composition to a patient. Such other components typically are known as excipients and the types and amounts to be used are within the skill of the art. The therapeutic pharmaceutical compositions consisting essentially of an anticholinergic agent and a sedative agent may contain one or more excipients. The excipients may provide desired delivery characteristics, depending on the route of preparation of the therapeutic pharmaceutical composition and the intended method of administration thereof to a patient. Such excipients specifically may include disintegrants, lubricants, diluents, binders, and/or coloring agents, among others, so long as the excipients do not materially affect the basic and novel characteristics of the therapeutic pharmaceutical compositions consisting essentially of an anticholinergic agent and a sedative agent. Suitable excipients include, for example, starches such as partially pregelatinized maize starch, other pregelatinized starch, or celluloses, suitable lubricants such as magnesium stearate, calcium stearate, talc or stearic acid and/or suitable diluents including lactose, among others. Any coloring agent certified by the FDA may be used, such as FD&C Yellow #6, among others.

The therapeutic pharmaceutical compositions consisting essentially of an anticholinergic agent and a sedative agent may be prepared using methods known in the pharmaceutical art. Such methods of preparation, for example, may include a direct compression method, a dry granulation method, wet granulation or encapsulation techniques.

The therapeutic pharmaceutical compositions generally are administered systemically and may be administered in various ways known in the art. Preferably, the compositions are provided to the patient by oral administration. Typically, the composition will be provided in tablet or capsule form. The composition may be provided in an immediate release form or formulated to provide sustained release of the blend of anticholinergic agent and sedative agent. In a preferred embodiment, the therapeutic pharmaceutical composition is prepared as a powder in an immediate release formulation and provided in capsule form for administration to the patient.

The amount of anticholinergic agent and sedative agent in the compositions or pharmaceutical formulations administered to a patient may vary depending upon multiple factors including, but not limited to, the particular composition, the patient's degree of illness, the patient's weight, and the patient's age. The patient may be any mammal in need of treatment for a gastrointestinal disorder or symptom of a gastrointestinal disorder. Preferably, the patient is a human patient.

The pharmaceutical compositions consisting essentially of an anticholinergic agent and a sedative agent may be used in the treatment of one or more gastrointestinal disorders or conditions or one or more symptoms thereof. In one embodiment, the therapeutic pharmaceutical compositions may be used to treat conditions or disorders requiring an antispasmodic effect. In a preferred embodiment, the therapeutic pharmaceutical compositions are used to treat ulcers or irritable bowel syndrome or symptoms of those disorders.

### EXAMPLE

The invention will be further explained by the following illustrative example that is intended to be non-limiting.

Capsules (100,000) were prepared containing methscopolamine nitrate and chlordiazepoxide hydrochloride according to the following formulation: methscopolamine nitrate, USP, 2.60 mg per dose (4.0% excess, 0.23 kg actual amount), 5.00 mg per dose chlordiazepoxide, 160.00 mg lactose monohydrate, 5.00 mg talc, USP, 30.00 mg Starch 1500®. The total weight was 202.6 mg.

While the invention has been described in detail and with reference to specific embodiments thereof, it will be apparent to one skilled in the art that various changes and modifications can be made without departing from the spirit and scope of the invention.

What is claimed is:

1. A therapeutic pharmaceutical composition consisting essentially of a therapeutically effective amount of a blended mixture of an anticholinergic agent comprising methscopolamine nitrate and a sedative agent comprising chlordiazepoxide hydrochloride.

2. The therapeutic pharmaceutical composition of claim 1, wherein the ratio of the methscopolamine nitrate to the chlordiazepoxide hydrochloride is about 0.5:1 to about 1:1.

3. The therapeutic pharmaceutical composition of claim 1, wherein the methscopolamine nitrate is present in an amount of about 2.0 mg to about 5.0 mg.

4. The therapeutic pharmaceutical composition of claim 1, wherein the therapeutic pharmaceutical composition further includes one or more pharmaceutical excipients.

5. The therapeutic pharmaceutical composition of claim 4, wherein the therapeutic pharmaceutical composition includes lactose, talc and pregelatinized starch.

6. The therapeutic pharmaceutical composition of claim 1, wherein after administration of the therapeutic pharmaceutical composition to a patient, substantially no liver toxicity is observed in the patient.

7. A therapeutic pharmaceutical composition consisting essentially of a therapeutically effective amount of a blended mixture of an anticholinergic agent comprising methscopolamine nitrate and a sedative agent comprising diazepam.

8. The therapeutic pharmaceutical composition of claim 7, wherein the methscopolamine nitrate is present in an amount of about 2.0 to about 5.0 mg and the diazepam is present in an amount of about 2.0 to about 5.0 mg.

9. The therapeutic pharmaceutical composition of claim 7, wherein the pharmaceutical composition further includes one or more pharmaceutical excipients.

10. The therapeutic pharmaceutical composition of claim 9, wherein the pharmaceutical composition includes lactose, talc and pregelatinized starch.

11. The therapeutic pharmaceutical composition of claim 7, wherein after administration of the therapeutic pharmaceutical composition to a patient, substantially no liver toxicity is observed in the patient.

12. A therapeutic pharmaceutical composition consisting essentially of a therapeutically effective amount of a blended mixture of an anticholinergic agent comprising methscopolamine nitrate and a sedative agent comprising phenobarbital.

13. The therapeutic pharmaceutical composition of claim 12, wherein the methscopolamine nitrate is present in an amount of about 2.0 to about 5.0 mg and the phenobarbital is present in an amount of about 16.0 to about 32.0 mg.

14. The therapeutic pharmaceutical composition of claim 12, wherein the pharmaceutical composition further includes one or more pharmaceutical excipients.

15. The therapeutic pharmaceutical composition of claim 14, wherein the pharmaceutical composition includes lactose, talc and pregelatinized starch.

16. The therapeutic pharmaceutical composition of claim 12, wherein after administration of the therapeutic pharmaceutical composition to a patient, substantially no liver toxicity is observed in the patient.

17. A therapeutic pharmaceutical composition consisting essentially of a therapeutically effective amount of a blended mixture of methscopolamine nitrate and chlordiazepoxide hydrochloride wherein the ratio of the methscopolamine nitrate to the chlordiazepoxide is about 0.5:1 to about 1:1.

18. The therapeutic pharmaceutical composition of claim 17, wherein the blended mixture further contains lactose, talc and partially pregelatinized maize starch.

19. The therapeutic pharmaceutical composition of claim 17, wherein the blended mixture is a powder for immediate release.

20. The therapeutic pharmaceutical composition of claim 17, wherein the blended mixture is in a sustained release formulation.

21. A therapeutic pharmaceutical composition consisting essentially of a therapeutically effective amount of methscopolamine nitrate for oral administration in an amount of about 2.0 to about 5.0 mg per dose.

22. The therapeutic pharmaceutical composition of claim 21, wherein the methscopolamine nitrate is provided in an oral formulation for immediate release.

23. The therapeutic pharmaceutical composition of claim 21, wherein the methscopolamine nitrate is in a sustained release formulation which provides an amount of about 8.0 mg to about 20.0 mg methscopolamine nitrate per day.

24. A method of alleviating at least one gastrointestinal disorder or at least one symptom of a gastrointestinal disorder in a human patient, the method comprising administering to the patient a therapeutic pharmaceutical composition consisting essentially of a therapeutically effective amount of a blended mixture of methscopolamine nitrate and chlordiazepoxide hydrochloride.

25. The method of claim 24, wherein the ratio of the methscopolamine nitrate to the chlordiazepoxide is about 0.5:1 to about 1:1.

26. The method of claim 24, wherein the therapeutic pharmaceutical composition is administered orally in an immediate release form four times a day.

27. The method of claim 24, wherein the therapeutic pharmaceutical composition is administered orally in a sustained release formulation given less than four times a day.

28. The method of claim 24, wherein the methscopolamine nitrate is present in an amount of about 2.0 mg to about 5.0 mg.

29. The method of claim 24, wherein the gastrointestinal disorder is an ulcer or irritable bowel syndrome.

30. The method of claim 24, wherein after administering the therapeutic pharmaceutical composition to the patient substantially no liver toxicity is observed in the patient.

31. A method of alleviating at least one gastrointestinal disorder or at least one symptom of a gastrointestinal disorder in a human patient, the method comprising administering to the patient a therapeutic pharmaceutical composition consisting essentially of a therapeutically effective amount of a blended mixture of methscopolamine nitrate and diazepam.

32. The method of claim 31, wherein the methscopolamine nitrate is present in an amount of about 2.0 to about 5.0 mg and the diazepam is present in an amount of about 2.0 to about 5.0 mg.

33. The method of claim 31, wherein the therapeutic pharmaceutical composition is administered orally in an immediate release form four times a day.

34. The method of claim 31, wherein the therapeutic pharmaceutical composition is administered orally in a sustained release formulation given less than four times a day.

35. The method of claim 31, wherein the gastrointestinal disorder is an ulcer or irritable bowel syndrome.

36. The method of claim 31, wherein after administering the therapeutic pharmaceutical composition to the patient substantially no liver toxicity is observed in the patient.

37. A method of alleviating at least one gastrointestinal disorder or at least one symptom of a gastrointestinal disorder in a human patient, the method comprising administering to the patient a therapeutic pharmaceutical composition consisting essentially of a therapeutically effective amount of a blended mixture of methscopolamine nitrate and phenobarbital.

38. The method of claim 37, wherein the methscopolamine nitrate is present in an amount of about 2. to about 5.0 mg and the phenobarbital is present in an amount of about 16.0 to about 32.0 mg.

39. The method of claim 37, wherein the therapeutic pharmaceutical composition is administered orally in an immediate release form four times a day.

40. The method of claim 37, wherein the therapeutic pharmaceutical composition is administered orally in a sustained release formulation given less than four times a day.

41. The method of claim 37, wherein the gastrointestinal disorder is an ulcer or irritable bowel syndrome.

42. The method of claim 37, wherein after administering the therapeutic pharmaceutical composition to the patient substantially no liver toxicity is observed in the patient.

43. A method of alleviating at least one gastrointestinal disorder or at least one symptom of a gastrointestinal disorder in a human patient, the method comprising administering to the patient a therapeutic pharmaceutical composition consisting essentially of a therapeutically effective amount of methscopolamine nitrate.
